(19) United States
(12) Patent Application Publication
(10) Pub. No.: US 2004/0054343 A1
(43) Pub. Date: Mar. 18, 2004

(54) Horizontal density gradient absorbent system for personal care products

(76) Inventors: Larry N. Barnett, Kingsport, TN (US); Jaime Braverman, Atlanta, GA (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/245,967

(22) Filed: Sep. 18, 2002

(51) Int. Cl. A61F 13/15; A61F 13/20

(52) U.S. Cl. 604/378

(57) ABSTRACT

An absorbent core for a personal care product such as an incontinence or catamenial product has a retention layer having major plane surfaces, a thickness, and a central target area positioned in an area most likely to receive insult from the discharge of bodily fluids. The retention layer has a horizontal density gradient from a lower density and higher permeability throughout the thickness of the target area to a higher density and lower permeability distal from the target area, thereby permitting faster fluid flow through the target area than through the surrounding higher density material. The retention layer is further covered by high-rate fluid acquisition and distribution material on both major plane surfaces. Thus, bodily fluids will be quickly drawn away from the skin of the wearer and rapidly distributed to both major plane surfaces and to the interior of the retention layer through its thickness and retained in the high absorbent capacity areas of the retention layer. The flow pattern through the retention layer thus increases the area available for free liquid to contact unwetted absorbent, such as superabsorbent material, without producing fluid blockage due to swelling of the absorbent.

### BACKGROUND OF THE INVENTION

Personal care products typically are made with a top sheet material, also referred to as a liner, an absorbent core and a liquid impervious back sheet. Some may also have a so-called “surge” layer for rapid fluid acquisition and, in some instances, distribution, or other specialized layers between the top sheet and absorbent core. Absorption of fluid, comfort and avoidance of leakage are the functions desired.

Absorbent products, such as the personal care products discussed herein in conjunction with the present invention, should have as little leakage as possible and deliver comfort and discretion to the user, e.g., such as by most efficiently utilizing its absorption capacity. Current personal care products may have relatively high leakage and thus offer only modest protection to the consumer. Leakage may be categorized by three key causes: fluid does not absorb into the product, fluid is absorbed into the product but subsequently leaves it, or fluid never contacts the product.

The specific reasons for leakage may be expressed further in terms of definitive mechanisms. A product, for instance, may not have suitable space for absorption due to localized saturation or low contact area. The product may not have a suitable driving force for absorption because the structure does not have the right balance of permeability and capillarity or wettability. The interfiber spaces may be partially full of fluid. Fluid may contact the product and run-off. The fluid may be too viscous or the pores or interfiber spaces are not large enough to allow fluid to pass through to the subjacent layer.

In all cases, the material systems and the concentration of materials in a specific product design may impact leakage by affecting intake, distribution, retention and transfer of fluids.

Intake includes the initial absorption of fluid into a dry product as well as the continued uptake of that fluid into the absorbent structure. The desirable product is one which absorbs fluid rapidly without releasing back the absorbed fluid to the user's skin. Development of superior intake systems requires an understanding of environmental conditions including the nature of the fluid and its discharge. Developing functional intake structures requires an understanding of material characteristics and their interaction with the fluid as components and systems of components including interfaces and product design.

Two interdependent factors in the intake and retention of bodily fluids are the area available for liquid-to-absorbent material contact, and the rate at which the liquid-to-absorbent material contact occurs. A third factor may be the overall amount or capability of absorption and liquid retention available from the absorptive material.

Typical absorbent cores, especially for urine absorption, often include a mixture of pulp and superabsorbent materials. The superabsorbent materials are slower to absorb, but have more absorption capacity, than pulp. A known phenomena called “gel blocking” interferes with the efficient use of superabsorbents. In gel blocking, the initial contact of liquid to superabsorbent material causes the superabsorbent material to swell, thus blocking further fluid movement and slowing the rate at which additional liquid may contact the unabsorbed superabsorbent material.

Various techniques have been proposed in the art to make personal care products such as diapers or other absorbent garments more efficient. The proposed techniques often suggest multiple layers of materials with each layer having a specialized construction or function. However, the construction of garments with specialized layers, which may be functionally very efficient, may also lead to escalating product costs due to the expense of making and placing the specialized layers together in a garment. Thus, it is desirable that fluid handling, or distribution, layers and fluid absorbent, or retention, layers be incorporated functionally into the personal care products in an easily manufactured and economical fashion while increasing absorbent core utilization.

There remains a need for a personal care product that is able to control and contain body exudates in such a way as to keep the wearer comfortable and protected from liquid being held near the skin, by rapid distribution and absorption of liquid. There is also a need for components for such products which are relatively inexpensive and highly effective.

### SUMMARY OF THE INVENTION

In response to the discussed difficulties and problems encountered in the prior art, a new absorbent core has been designed as a system to provide enhanced fluid intake, distribution and retention functions. A composite absorbent core according to the present invention will generally include a retention layer having major plane surfaces, a thickness, and a central target area positioned in the area most likely to receive insult from the discharge of bodily fluids. The retention layer has a horizontal density gradient in its material from the target area, having lower density and higher permeability throughout the thickness of the target area, to higher density and lower permeability areas distal from the target area. Thus, the target area permits faster fluid flow-through than the surrounding higher density material. In certain aspects of the invention, embodiments may have wettability gradients in the absorbent core in which less wettable components are placed near the target area and more wettable components are placed at, or near to, edges of high absorbency and retention materials.

The retention layer is further covered by high-rate fluid acquisition and distribution layers on both its major plane surfaces. Thus, bodily fluids will be quickly drawn away from the skin of the wearer and rapidly distributed to both major plane surfaces and to the interior of the retention layer throughout its thickness and retained in the high capacity areas of the retention layer. The flow pattern through, and around, the retention layer thus increases the area available for free liquid to contact unwetted absorbent, such as superabsorbent material, without producing fluid blockage due to swelling of the absorbent.

Additional horizontal material gradients, especially of high capacity absorbent materials, are provided in conjunction with the low density target area to aid the liquid distribution and retention functions. Different acquisition and distribution layer types may be provided for the upper major surface of the retention layer closest to the wearer and lower major surface of the retention layer away from the wearer to aid in the distribution of fluid. All such gradients may be made inherent in the layers of the absorbent core through the original placement of materials within the layers. Alternatively, the gradients may be later induced by manipulation of, or addition to, the materials, or combinations thereof.

Personal care products using this composite are also contemplated to be within the scope of this invention. One such personal care product has a liquid impermeable backsheet, a liquid permeable topsheet, or liner, and the absorbent core, with a horizontal density gradient, located between the topsheet and backsheet. The horizontal density gradient absorbent core, according to one aspect of the present invention, can be a composite structure of upper and lower acquisition and distribution layers surrounding the retention layer and having major surfaces in the X-Y plane, or horizontal direction, and a depth, or thickness, in the Z direction that is suitable for use as a highly effective and inexpensively made fluid intake, distribution and retention layer in a disposable absorbent article.

Horizontal gradients may, in certain schemes of process and production, be more economical and efficacious than vertical gradient systems. For example, coordinating the timing of the material deposition onto a forming wire, and if desirable, having a controlled area vacuum for the wire, material deposition can be controlled in at least one of a machine direction or a cross direction of a nonwoven web. Thus the retention layer may be provided with material gradients in at least one of the machine direction or the cross direction and may be customized according to the specific needs for a single composite structure in accordance with the present invention. The acquisition and distribution layers can add vertical gradients by placement of separate surge layers over and under the retention layer.

The absorbent core can provide a horizontal density gradient of superabsorbent materials. The absorbent core may further contain other gradients of materials or types to aid in the fluid intake, distribution, and retention functions. For example, various gradients can be created in the retention layer by selectively mixing fibers, including natural and synthetic fibers, with different properties such as denier, fiber composition, fiber morphology, or combinations thereof to aid in the rate of intake and absorption of liquid. The retention layer may utilize surfactants or additives in the absorptive material, e.g., the pulps, which may be added at various times in the forming processes. Superabsorbent polymers having different absorption properties may be selectively placed in the retention layer, as may mixtures of different pulps. Calender rolls may mechanically “print” a selected density pattern into the absorbent core.

Other aspects of the invention may have intermixed layers of synthetic materials that provide either density or wettability gradients or both. Other aspects of the invention may include using hot melts or latex adhesives within, or in combination with, the absorbent core in order to increase the absorbent core integrity while at the same time modifying the surface energy of the material. By selectively applying these hot melts or latex adhesive binders, preferential fluid passageways can be created, increasing the fluid movement to designated areas.

According to some aspects of the present invention, the acquisition and distribution layers surrounding the retention layer may be modified to provide enhanced functionality of liquid distribution and absorption. The acquisition and distribution layers may be further modified by changing their fiber types, or combinations of any of their composition, basis weight, and surfactant types. Latex or hot melt binders, or other attachment means, may be used to bond these acquisition and distribution layers to the retention layer to improve fluid transport according to the functionality dictates of the particular application of the invention.

The acquisition and distribution layers of this invention can be made, e.g., by the through air bonding of multiple layers. The layers can be homogenous or heterogeneous. In the heterogeneous case, any or all of the fiber denier, fiber type or surfactant used can be different from the top layer to the bottom layer and may create a pore size gradient, or wettability gradient, or both. These gradients may improve the fluid absorption and reduce rewet, i.e., expulsion of previously absorbed fluids.

### BRIEF DESCRIPTION OF THE DRAWINGS

The accompanying drawings are presented as an aid to explanation and understanding of various aspects of the present invention only and are not to be taken as limiting the present invention. The drawings are not necessarily to scale, nor should they be taken as photographically accurate depictions of real objects unless otherwise stated.

FIG. 1 is a representative plan view of an absorbent article, specifically an exemplary diaper, laid flat with the interior of the diaper facing the viewer, and with cut-away portions showing various layers of the article.

FIG. 2 is a cross sectional side view of an absorbent core indicating the horizontal gradient from a lower density target area to the higher density distal areas in the absorbent core.

FIG. 3 is a top plan view of an absorbent core indicating the horizontal gradient from a lower density target area to the higher density distal areas in the absorbent core.

FIG. 4 is a cross sectional side view of an absorbent core indicating the flow path created to increase the liquid-to-absorbent material contact area.

FIG. 5 is a cross sectional side view of an absorbent core indicating the horizontal gradient from a higher permeability and lower absorption capacity target area to the lower permeability and higher absorption capacity distal areas in the absorbent core.

FIG. 6 is a cross sectional side view of an absorbent core indicating different types of superabsorbent material horizontal gradients on the right side of the Figure and the left side of the Figure.

FIG. 7 is a cross sectional side view of an absorbent core indicating a pulp type horizontal gradient on the right side of the Figure and various materials of the retention layer on the left side of the Figure.

FIG. 8 is a cross sectional side view of an absorbent core indicating different pore sizes between the various layers of the absorbent core on the right side and the left side of the Figure.

FIG. 9 is a cross sectional side view of an absorbent core indicating different levels of wettability between the various layers of the absorbent core on the right side and the left side of the Figure.

### DEFINITIONS

“Disposable” includes being disposed of after a single, or limited, use and not intended to be washed and reused.

A “layer” is defined as a generally recognizable combination of similar material types or function existing in the X-Y plane.

The “upward” or “top” position layers are closer to the body of a wearer than “downward” “lower” or “bottom” layers when the article is worn.

“Composite” is defined as having two or more discrete components.

As used herein and in the claims, the term “comprising” is inclusive or open-ended and does not exclude additional uncredited elements, compositional components, or method steps.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, airlaying processes and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

“Spunbond fibers” refers to small diameter fibers that are formed by extruding molten thermoplastic material as filaments from a plurality of fine capillaries of a spinneret. Such a process is disclosed in, for example, U.S. Pat. No. 3,802,817 to Matsui et al., U.S. Pat. No. 4,340,563 to Appel et al. The fibers may also have shapes such as those described, for example, in U.S. Pat. No. 5,277,976 to Hogle et al. which describes fibers with unconventional shapes.

“Meltblown fibers” as used herein refers to fibers formed by extruding a molten thermoplastic material through a plurality of fine, usually circular, die capillaries as molten threads or filaments into converging high velocity, usually hot, gas (e.g., air) streams which attenuate the filaments of molten thermoplastic material to reduce their diameter, which may be to microfiber diameter. Thereafter, the meltblown fibers are carried by the high velocity gas stream and may be deposited on a collecting surface to form a web of randomly disbursed meltblown fibers. Such a process is disclosed, for example, in U.S. Pat. No. 3,849,241 to Butin et al. Meltblown fibers are microfibers which may be continuous or discontinuous, are generally smaller than 10 microns (μm) in average diameter, and are generally tacky when deposited onto a collecting surface.

“Airlaying” is a well-known process by which a fibrous nonwoven layer can be formed. In the airlaying process, bundles of small fibers having typical lengths ranging from about 1 to about 19 millimeters (mm) are separated and entrained in an air supply and then deposited onto a forming screen, usually with the assistance of a vacuum supply. The randomly deposited fibers then are bonded to one another using, for example, hot air, water compaction, or a spray adhesive. Airlaying is taught in, for example, U.S. Pat. No. 4,640,810 to Laursen et al. Air laying may include coform deposition which is a known variant wherein pulp or other absorbent fibers are deposited in the same air stream onto the forming screen. The screen may also be referred to herein as a forming wire.

As used herein, “through-air bonding” means a process of bonding a nonwoven bicomponent fiber web in which air which is sufficiently hot to melt one of the polymers of which the fibers of the web are made is forced through the web. The melting and resolidification of the polymer provides the bonding. In the through-air bonder, air having a temperature above the melting temperature of one component and below the melting temperature of another component may be used. The hot air melts the lower melting polymer component and thereby forms bonds between the filaments to integrate the web.

“Personal care product” means diapers, wipes, training pants, absorbent underpants, adult incontinence products, feminine hygiene products, wound care items like bandages, and other articles.

Words of degree, such as “about,” “substantially,” and the like are used herein in the sense of “at, or nearly at, when given the manufacturing and material tolerances inherent in the stated circumstances” and are used to prevent the unscrupulous infringer from unfairly taking advantage of the invention disclosure where exact or absolute figures are stated as an aid to understanding the invention.

As used herein, the term “machine direction” means the length of a fabric in the direction in which it is produced. The term “cross direction” or “cross machine direction” means the width of fabric, i.e. a direction generally perpendicular to the machine direction.

“Density” as used herein refers to the distribution of a quantity or type per unit of space and is not limited to defining the mass of the object.

“Gradient” refers to a change of material composition or concentration, or both.

“Pore size” refers to openings in a material layer and may include voids or channels of irregular or regular shape such as between fibers of a fibrous nonwoven web or voids in a film.

“Calender” refers to compression between a roller and another hard surface. The calender roll or rolls may be shaped to permit selective compression of the material.

### DETAILED DESCRIPTION OF THE PRESENTLY PREFERRED EMBODIMENTS

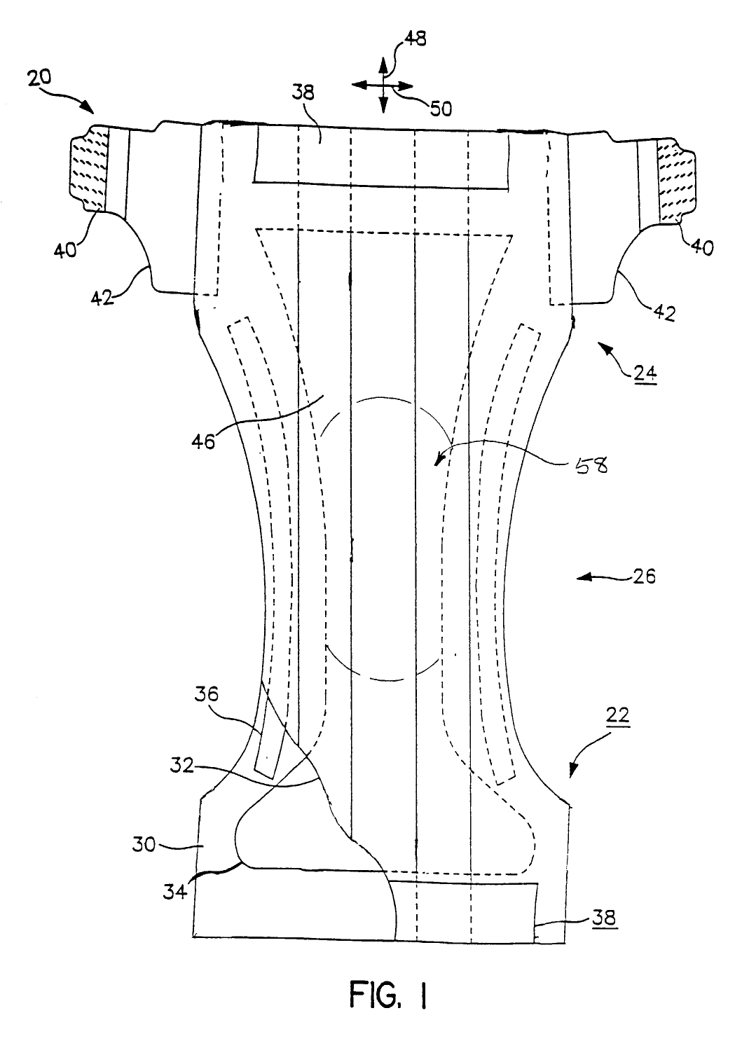

FIG. 1 is a representative plan view of an absorbent article, such as disposable diaper 20, in its flat-out, or unfolded state. Portions of the structure are partially cut away to more clearly show the interior construction of diaper 20. The surface of the diaper 20 which contacts the wearer is facing the viewer.

With reference to FIG. 1, the disposable diaper 20 generally defines a front waist section 22, a rear waist section 24, and an intermediate section 26 which interconnects the front and rear waist sections. The front and rear waist sections 22 and 24 include the general portions of the diaper which are constructed to extend substantially over the wearer's front and rear abdominal regions, respectively, during use. The intermediate section 26 of the diaper includes the general portion of the diaper that is constructed to extend through the wearer's crotch region between the legs. Thus, the intermediate section 26 generally includes the target zone, or area, 58 where repeated liquid surges, or insults, typically occur in the diaper.

The diaper 20 includes, without limitation, an outer cover, or back sheet 30, a liquid permeable bodyside liner, or topsheet, 32 positioned in facing relation with the back sheet 30, and an absorbent core 34, which is a composite liquid acquisition and retention structure according to the present invention as further explained below, and located between the back sheet 30 and the topsheet 32.

The back sheet 30 defines a length, or longitudinal direction 48, and a width, or lateral direction 50 which, in the illustrated embodiment, coincide with the length and a width of the diaper 20. The absorbent core 34 generally has a length and width that are less than the length and width of the back sheet 30, respectively. Thus, marginal portions of the diaper 20, such as marginal sections of the back sheet 30, may extend past the terminal edges of the absorbent core 34. In the illustrated embodiments, for example, the back sheet 30 extends outwardly beyond the terminal marginal edges of the absorbent core 34 to form side margins and end margins of the diaper 20. The topsheet 32 is generally coextensive with the back sheet 30 but may optionally cover an area which is larger or smaller than the area of the back sheet 30, as desired.

The diaper 20 may include leg elastics 36 which are constructed to operably tension the side margins of the diaper 20 to provide elasticized leg bands which can closely fit around the legs of the wearer to reduce leakage and provide improved comfort and appearance. Waist elastics 38 are employed to elasticize the end margins of the diaper 20 to provide elasticized waistbands. The waist elastics 38 are configured to provide a resilient, comfortably close fit around the waist of the wearer.

In the illustrated embodiment, the diaper 20 includes a pair of side panels 42 to which fasteners 40, indicated as the hook portion of a hook and loop fastener, are attached. Other fastener attachment means such as adhesive tapes, etc. may be also used. Generally, the side panels 42 are attached to the side edges of the diaper 20 in one of the waist sections 22, 24 and extend laterally outward therefrom. The side panels 42 may be elasticized or otherwise rendered elastic. For example, the side panels 42, or indeed, any precursor component webs of the garment, may be an elastomeric material.

As representatively illustrated in FIG. 1, the disposable diaper 20 may also include a pair of containment flaps 46 which are configured to provide a barrier to the lateral flow of body exudates. The containment flaps 46 may be located along the laterally opposed side edges of the diaper 20 adjacent the side edges of the liquid retention structure 34. Each containment flap 46 typically defines an unattached edge which is configured to maintain an upright, perpendicular configuration in at least the intermediate section 26 of the diaper 20 to form a seal against the wearer's body.

At least certain layers of a composite absorbent core according to this invention, may be made using the air laid process. The production of air laid nonwoven composites is well defined in the literature and documented in the art. Examples include the Dan-Web process as described in U.S. Pat. No. 4,640,810 to Laursen et al., the Kroyer process as described in U.S. Pat. No. 4,494,278 to Kroyer et al. and U.S. Pat. No. 5,527,171 to Soerensen; the method of U.S. Pat. No. 4,375,448 to Appel et al. or other similar methods.

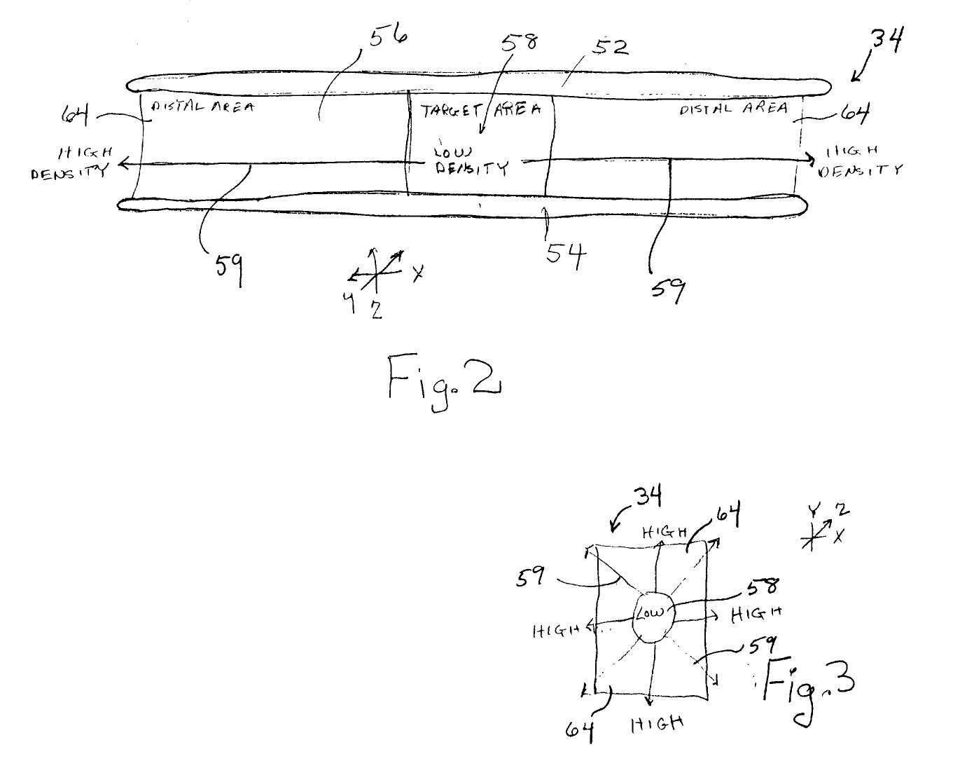

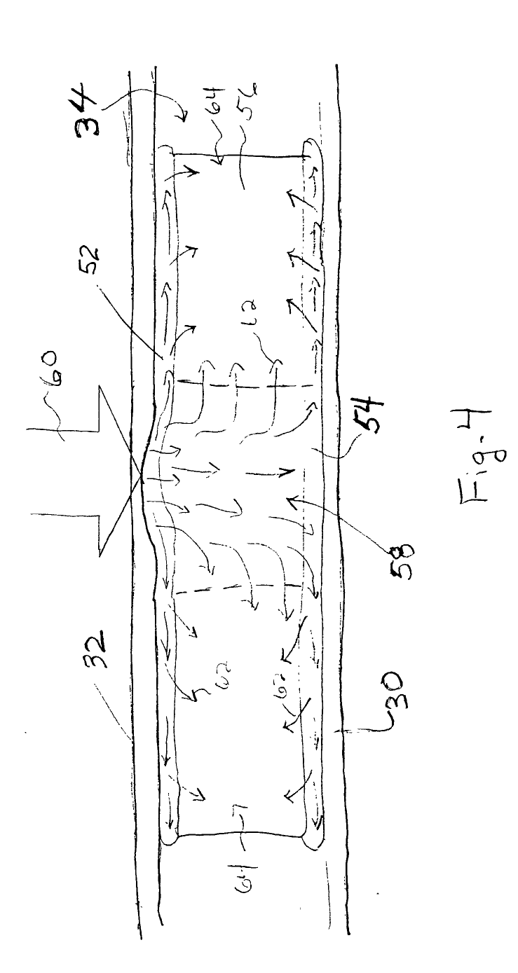

Referencing FIG. 2, the absorbent core 34 generally has denominated an upper acquisition and distribution layer 52 and a lower acquisition and distribution layer 54 wherein the upper acquisition and distribution layer 52 is the acquisition and distribution layer closer to the body of a wearer while the personal care product is in use. A retention layer 56 is located between the upper acquisition and distribution layer 52 and the lower acquisition and distribution layer 54 and may have various gradients in the horizontal direction, i.e. in the major, or X-Y, plane of the layer. The major axes of the web will be indicated in the drawings where appropriate, with the thickness being indicated in the Z-direction, the X axis being indicated as the machine direction (MD) and the Y axis being indicated as the cross, or cross machine, direction (CD) for ease of explanation. The gradients may include, e.g., having a gradient of increasing density outward from a centrally located target area 58 as indicated by the arrows 59 in the indicated X axis of both FIGS. 2 and 3. The target 58 area will generally be referenced to that area of a garment, e.g. 20, most likely to receive surging insults of bodily fluid discharge. Wettability and permeability gradients may further exist in the X-Y plane.

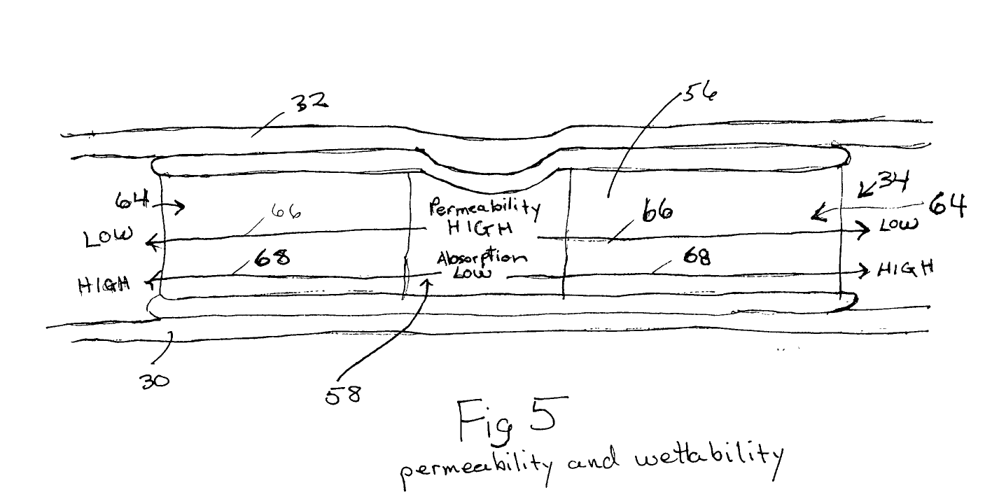

Referencing FIG. 4, the absorbent core 34 is located between the liner 32 and the backsheet 30. The upper acquisition and distribution layer 52 is located adjacent the liner 32 and the lower acquisition and distribution layer 54 is located adjacent the backsheet 34. A bodily fluid insult, represented by the large arrow 60, contacts the liner 32 generally in the target area 58 of the retention layer 56 of the absorbent core 34. The fluid of the insult will flow through the absorbent core 34 as indicated by the smaller arrows, collectively 62. The fluid will travel rapidly along the upper acquisition and distribution layer 52 away from the initial point of insult and towards the distal areas 64 of the retention layer 56 in which the higher density material having a higher absorption capacity, e.g., a higher concentration of superabsorbent material is located. The fluid will also travel rapidly through the low density, high permeability material of the target area 58 and be carried away from the skin of the wearer located next to the liner 32. The fluid which has traveled through the Z-direction thickness of the retention layer 56 will also be transported away from the target area towards the distal areas 64 by the lower acquisition and distribution layer 54. By constructing the absorbent core 34 according to the present invention, the area available for liquid to absorbent material contact and the rate at which the liquid contacts the absorbent material are both increased, while gel blocking is avoided, resulting in improved functionality for the absorbent core.

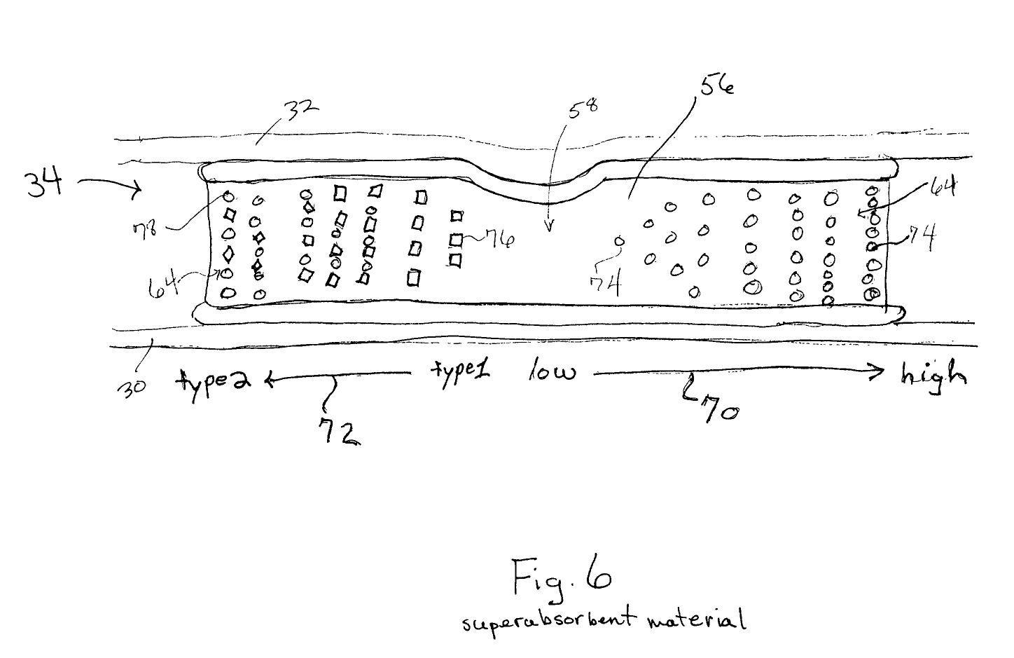

Referencing FIG. 5, the retention layer 56 is shown with a gradient of permeability indicated by arrows 66 from high permeability near the low density target area 58 to low permeability near the high density distal areas 64. Also, as indicated by arrows 68, the retention layer 56 is shown with a gradient of permeability indicated from low absorption near the low density target area 58 to high absorption near the high density distal areas 64.

Referencing FIG. 6, the absorbent core 34 indicates on the right side thereof a horizontal gradient, as indicated by arrow 70, of the retention layer 56 from a lower density of superabsorbent material 74 near the target area 58 to a higher density of superabsorbent material 74 in the distal areas 64. The indicated gradient may promote better fluid management by preventing gel blocking due to initial swelling of the superabsorbent material 74 from contact with the initial fluid traveling through the low density target area which might block later fluid distribution. It will be appreciated by the person having ordinary skill in the art that, although not shown as such, the target area 58 may contain some superabsorbent material. On the left side of FIG. 6, the change in densities is from a higher density of a first type of superabsorbent material 76 near the target area 58 to a higher density of a second type of superabsorbent material 78 in the distal areas. The indicated gradient may be used to prevent gel blocking by using a slower swelling superabsorbent material for the first type superabsorbent material 76 to prevent a rapid initial swelling of the superabsorbent material 74 due to initial fluid contact travelling through the low density target area 58 which might block later fluid distribution. A higher capacity, or faster swelling, superabsorbent material may be used as the second superabsorbent material type 78 in the distal areas 64 where gel blocking is not a large impediment to the rate or contact area of fluid transfer from free liquid to the absorbent material. Superabsorbents useful in or near the target area 58 may have a high gel strength and tend to have high gel bed permeability even when saturated. Examples of such superabsorbents are Favor® 9543 from Stockhausen, Greensboro, N.C. Superabsorbents useful in intermediate areas of the gradient may have moderate gel strength and tend to exhibit moderate swelling rates. Examples of such superabsorbents include Favor® 880 from Stockhausen, Greensboro, N.C. and Drytech® 2035 from Dow Chemical, Midland, Mich. Superabsorbents useful in the distal areas 64 effectively away from the target area 58 may include those having high fluid retention capacity and moderate to relatively high swelling rates. For example, smaller superabsorbent particles may be used to achieve the high swelling rate. Examples of some superabsorbents which may be useful in these areas include Favors 880 from Stockhausen, Greensboro, N.C. and Drytech® 2035 from Dow Chemical, Midland, Mich.

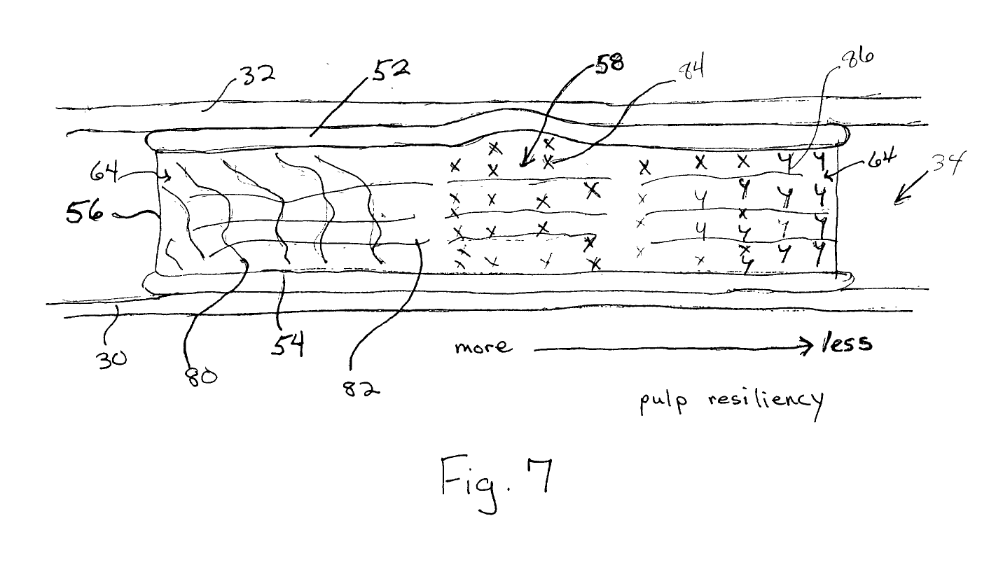

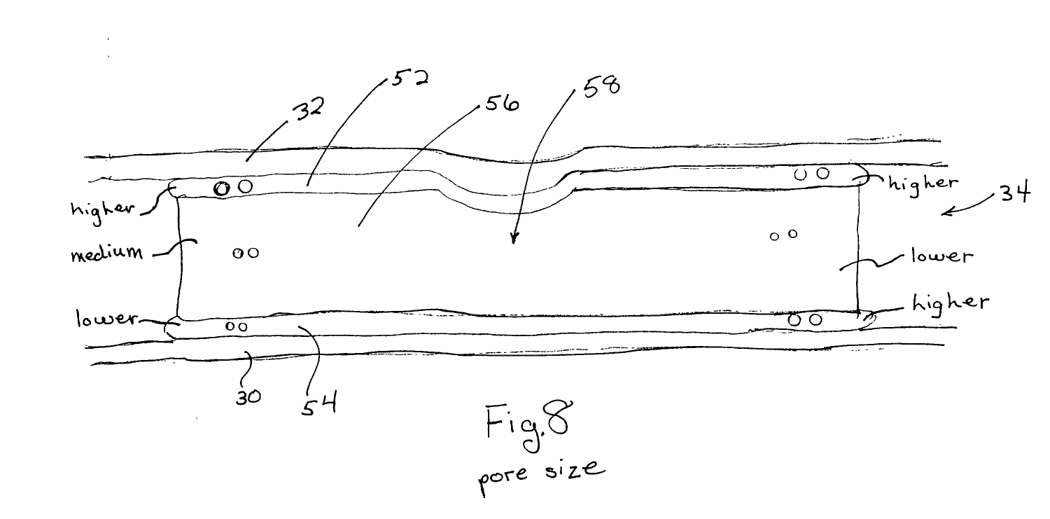

Referencing FIG. 7, a cross sectional side view of the absorbent core 34 indicates on the left side thereof the inclusion of synthetic fibers 80 and elastic fibers 82 within the composition of the retention layer 56; and on the right side thereof, the change in densities from a higher density of a first type of pulp fibers 84 in the target area 58 to a higher density of a second type of pulp fibers 86 in the distal areas 64 within the retention layer 56. The absorbent core 34 may include a nonwoven web of various synthetic fibers 80, e.g. thermoplastic fibers, for structural and binder purposes; as well as elastomeric fibers 82, which may also be thermoplastic, to facilitate expandability of the absorbent core and hence better conformance to a user's body. In other aspects of the invention, elastic filaments may be attached with a hot melt to the absorbent core 34 allowing it to be elongated and returned, at least to some extent, to its original position.

The first type of pulp fibers 84 in this aspect of the invention are a more resilient pulp fiber which is less subject to compaction and densification than the second type of pulp fibers 86. Thus, if the retention layer 56 or absorbent core 34 is subjected to calendering, the more resilient first type of pulp fibers 84 will ultimately retain a lower density for the target area than the less resilient second type of pulp fibers 86.

The synthetic fibers 80 may be used in these structures to help provide mechanical integrity and stabilization of the retention layer 56. Various known types of fibers such as meltblown and spunbond fibers may be used in certain aspects of the invention. These fibers may be assembled into the various layers of the absorbent core 34 to create various gradients by selectively mixing fibers, including natural and synthetic fibers, with different properties such as denier, fiber composition, fiber morphology, or combinations thereof. The layers may further utilize surfactants or additives in, or on, the various fibers during assembly of the layers prior to joining into the absorbent core 34, or may be post-treated with said surfactants or additives during conversion into the absorbent core or placement within the finished product structure.

Thermoplastic or other synthetic fibers useful in or near the target area 58 include those that have the ability to maintain an open structure. Such fibers may include, but are not limited to, polyethylene, polypropylene, polyethylene terephthalate (PET), Nylon 6, Nylon 66, acrylic fibers and lyocel fibers, as well as bicomponent fibers in various deniers. According to some aspects of the invention, synthetic fibers useful in intermediate areas of the gradient include those having a high level of wettability. Synthetic fibers, if present in the distal areas 64 located effectively away from the target area 58, should be highly wettable and of fine denier.

Binders may include fiber, liquid or other binder means which in some instances may be thermally activated. Preferred binder fibers for inclusion are those having a relatively low melting point such as polyolefin fibers. Lower melting point polymers provide the ability to bond the fabric together at fiber cross-over points upon the application of heat. Other suitable binders may include hot melt or latex adhesives which can increase the fluff integrity of the absorbent core or modify the absorption characteristics of the absorbent core, or both. Certain materials can be treated with appropriate surfactants to modify their absorption characteristics and aid in the fluid movement within the absorbent core.

Cellulosic wood pulps may include standard softwood fluffing grade such as CR-1654 (U.S. Alliance Pulp Mills, Coosa, Ala.), Weyerhaeuser NB416 southern softwood pulp, or Foley fluff from Buckeye Corporation Memphis, Tenn., which may be used as pulp material within the context of the present invention. Cellulose fibers for use in or near the target area 58 include those that can maintain an open structure when wetted, such as chemically cross linked, mercerized or otherwise stiffened fibers. Examples of such fibers include NHB416 from Weyerhaeuser, Tacoma, Wash. and HPF2 and HPZ3 from Buckeye, Memphis, Tenn. Pulp types that may be suitable for use in intermediate areas of the gradient include conventional southern softwood fibers such as CR54 from Alliance Forest Products, Coosa, Ala., NB416 from Weyerhaeuser, Tacoma, Wash., and Foley fluff from Buckeye, Memphis, Tenn. Pulp types useful in the distal areas 64 effectively away from the target area 58 are typically fine and highly wettable. Examples include hardwood fibers such as Eucalyptus, Sulfatate HJ from Rayonier, Jesup, Ga.

The target area may further have a higher concentration of synthetic fibers selected for increasing the permeability and fluid intake rate. Depending on the fiber selection, such fibers may also increase the material resiliency to at least partially maintain or recoup any changes of absorbent void volume due to changes in the wearer's body pressure against the article during the product wear.

Pulp may be modified in order to enhance the inherent characteristics of the fibers and their processability. Curl may be imparted to the fibers by methods including chemical treatment or mechanical twisting. Curl is typically imparted before crosslinking or stiffening. Pulps may be stiffened by the use of crosslinking agents such as formaldehyde or its derivatives, glutaraldehyde, epichlorohydrin, methylolated compounds such as urea or urea derivatives, dialdehydes such as maleic anhydride, non-methylolated urea derivatives, citric acid or other polycarboxylic acids. Some of these agents are less preferable than others due to environmental and health concerns. Pulp may also be stiffened by the use of heat or caustic treatments such as mercerization. Examples of these types of fibers include NHB416 which is a chemically crosslinked southern softwood pulp fiber with enhanced wet modulus, available from the Weyerhaeuser Corporation of Tacoma, Wash. Other useful pulps may include fully debonded pulp (NF405) and non-debonded pulp (NB416) and PH Sulfite pulp, also from Weyerhaeuser. HPZ3 from Buckeye Technologies, Inc. of Memphis, Tenn., has a chemical treatment that sets in a curl and twist, in addition to imparting added dry and wet stiffness and resilience to the fiber. Another suitable pulp is Buckeye HPF2 pulp and still another is IP SUPERSOFT® from International Paper Corporation.

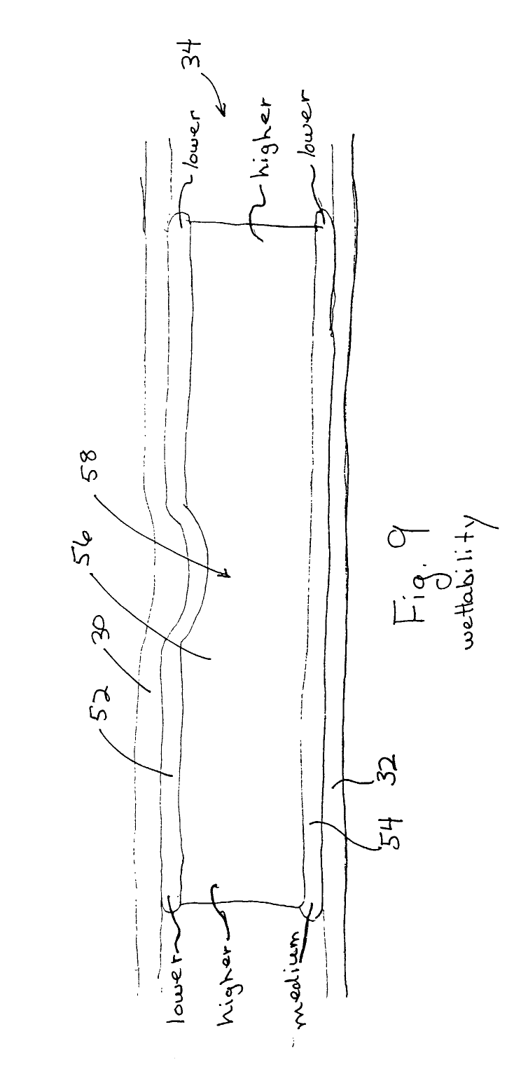

Referencing FIG. 8, a cross sectional side view of an absorbent core, on the right side thereof, the difference in pore sizes from a lower average pore size in the retention layer 56 to a higher average pore size in the acquisition and distribution layers 52, 54 surrounding the retention layer 56 is indicated. On the left side of the Figure, a change from higher average pore size in the upper acquisition and distribution layer 52, to an intermediate average pore size in the retention layer 56, to a lower average pore size in the lower acquisition and distribution layer 54, is illustrated. Pore size defines the voids in the nonwoven materials of the layers which create open flow channels facilitating the movement of liquid. By selecting the appropriate pore size differential between the layers 52, 54, 56 as indicated in FIG. 8, the rate of distribution of liquid through and to the various parts of the absorbent core 34 may be further controlled.

Upper and lower acquisition and distribution layers 52 and 54, respectively, can made with multiple layers to enhance fluid absorption, such as one of: the layers having different fiber types, different fiber treatments, and different basis weights as are known in the art, or combinations thereof. Further, layer 54, since is further away from the user's body, may have a smaller pore size than the upper layer 52, which may need to be more capable of quickly absorbing fluid. Additionally, the lower layer 54 can be more wettable than layer 52 to allowed allow better fluid distribution in the product's lower portion. Upper and lower acquisition and distribution layers 52 and 54 will generally have basis weights between about 20 and about 200 gsm, and desirably have basis weights between about 50 and about 150 gsm. It will be understood that the basis weight of the upper and lower acquisition and distribution layers need not be equivalent.

FIG. 9 is a cross sectional side view of an absorbent core indicating, on the right side thereof, the difference in wettability from a higher wettability in the retention layer 56 and a lower wettability in the acquisition and distribution layers 52, 54 surrounding the absorbent core; and on the left side, a change from lower wettability in the upper acquisition and distribution layer 52, to a higher wettability in the retention layer 56, to an intermediate wettability in the lower acquisition and distribution layer 54. By selecting the appropriate wettability differential between the layers 52, 54, 56 as indicated in FIG. 9, the rate of distribution of liquid through and to the various parts of the absorbent core 34 may be further controlled. Additionally, fibers of different wettability can be placed to create horizontal flow gradients.

Examples of suitable acquisition and distribution layers 52, 54, also sometimes referred to as surge management layers, are described in U.S. Pat. No. 5,486,166 to Bishop et al. and U.S. Pat. No. 5,490,846 to Ellis. Other examples of surge management layers include those described in U.S. Pat. No. 5,364,382 to Latimer et al., U.S. Pat. No. 5,490,846 to Ellis et al., U.S. Pat. No. 5,429,629 to Latimer et al., U.S. Pat. No. 5,509,915 to Hanson et al., U.S. Pat. No. 5,192,606 to Proxmire et al., and European Patent Application EP 0 539 703 A1, published May 5, 1993.

One method of making a retention layer of this invention is by the airlaying process using multiple spray heads which are timed and coordinated to aid in placing the various components at certain points along the structure of the web. This may occur in the machine direction where intermittent placement along the machine direction occurs as a function of time. Vacuum boxes may be so placed, or obstructed, as to aid in the selective deposition of the various materials on the forming wire, whether differentiated in their machine direction or cross direction spacing. Compaction, or calender, rolls, which may be heated, may be used to further control the density of the layers and to aid in bonding of the composite absorbent core. Further, it will be noted that, as illustrated, the target area may be made concave or convex towards the wearer. This may be accomplished through the use of patterned calendering as appropriate to initial distribution of material densities within the layers in order to maintain the preferred horizontal density gradient according to the present invention. Patterned calendering may be more efficient when performed as the absorbent core is placed in the product at the so-called “conversion line”, i.e., where the various layers are assembled into the commercial product.

As will be appreciated by those skilled in the art, changes and variations to the invention are considered to be within the ability of those skilled in the art. It will be appreciated by those of skill in the art that various materials, as well as their amounts, and types, may be utilized according to the present invention to adapt the absorbent core to a variety applications while remaining within the spirit of the present invention. Such changes and variations are intended by the inventors to be within the scope of the invention.

We claim:

1. An absorbent core for an absorbent article, comprising:
a) a retention layer having major plane surfaces, a thickness, and a target area for positioning in the absorbent article to receive insult from bodily fluids;
b) the retention layer having a horizontal density gradient from a first density in the target area to a second density distal from the target area; and
c) the retention layer being contacted by an upper fluid acquisition and distribution layer and a lower fluid acquisition and distribution layer on its major plane surfaces.

2. The absorbent core of claim 1, wherein the density varies by distribution of a particular type of matter per unit area.

3. The absorbent core of claim 1, wherein the density varies by mass per unit area.

4. The absorbent core of claim 1, wherein the density varies by distribution of a particular type of matter per unit area and mass per unit area.

5. The absorbent core of claim 1, wherein the first density is greater than the second density.

6. The absorbent core of claim 1, wherein the second density is greater than the first density.

7. An absorbent core for an absorbent article, comprising:
a) a retention layer having major plane surfaces, a thickness, and a target area for positioning in the absorbent article to receive insult from bodily fluids;
b) the retention layer having a horizontal density gradient from a lower density in the target area to a higher density distal from the target area; and
c) the retention layer being contacted by an upper fluid acquisition and distribution layer and a lower fluid acquisition and distribution layer on its major plane surfaces.

8. The absorbent core for an absorbent article of claim 7, wherein the absorbent core has a gradient consisting of at least one of permeability, wettability and absorption capacity in a horizontal direction.

9. The absorbent core for an absorbent article of claim 8, wherein the absorbent core has gradients of permeability and absorption capacity in a horizontal direction.

10. The absorbent core for an absorbent article of claim 8, wherein the absorbent core has gradients of permeability and wettability in a horizontal direction.

11. The absorbent core for an absorbent article of claim 10, wherein the retention layer is selectively treated to modify its wettability.

12. The absorbent core for an absorbent article of claim 7, further comprising: the retention layer having a superabsorbent material gradient from a low concentration of superabsorbent material in the target area to a higher concentration of superabsorbent material in areas distal from the target area.

13. The absorbent core for an absorbent article of claim 7, further comprising: the retention layer having a superabsorbent material gradient from a higher concentration of a first composition of superabsorbent material proximal to the target area to a higher concentration of a second composition of superabsorbent material in areas distal from the target area.

14. The absorbent core for an absorbent article of claim 7, wherein the retention layer further comprises thermoplastic fibers.

15. The absorbent core for an absorbent article of claim 7, wherein the retention layer further comprises elastomeric fibers.

16. The absorbent core for an absorbent article of claim 7, wherein the retention layer further comprises pulp fibers.

17. The absorbent core for an absorbent article of claim 7, wherein the retention layer further comprises at least one of a hot melt or latex adhesive.

18. The absorbent core for an absorbent article of claim 16, further comprising: the retention layer having a pulp fiber gradient from a higher concentration of a first composition of more resilient pulp fibers in the target area to a higher concentration of a second composition of less resilient pulp fibers in areas distal from the target area.

19. The absorbent core for an absorbent article of claim 7, wherein the upper and lower fluid acquisition and distribution layers have a larger average pore size than the retention layer.

20. The absorbent core for an absorbent article of claim 7, wherein the upper and lower fluid acquisition and distribution layers have a lower wettability than the retention layer.

21. The absorbent core for an absorbent article of claim 7, wherein the upper fluid acquisition and distribution layer has a larger average pore size than the retention layer; and
the lower fluid acquisition and distribution layer has a smaller average pore size than the upper fluid acquisition and distribution layer.

22. The absorbent core for an absorbent article of claim 7, wherein the upper fluid acquisition and distribution layer has a lower wettability than the retention layer, and
the lower fluid acquisition and distribution layer has a wettability intermediate to the upper acquisition and distribution layer and the retention layer.

23. The absorbent core for an absorbent article of claim 7, wherein at least one of the upper fluid acquisition and distribution layer and the lower fluid acquisition and distribution layer is made with multiple layers to enhance fluid absorption, including one of: the multiple layers having different fiber types, the multiple layers having different fiber treatments, and the multiple layers having different basis weights.

24. The absorbent core for an absorbent article of claim 7, wherein a density gradient does not exist through a thickness of the retention layer.

25. The absorbent core for an absorbent article of claim 7, wherein the target area is shaped such that it is concave away from a wearer of the article.

26. The absorbent core for an absorbent article of claim 25, wherein the target area is selectively calendered with respect to a non-target area of the retention layer.

27. The absorbent core for an absorbent article of claim 7, wherein the target area is shaped such that it is convex towards a wearer of the article.

28. The absorbent core for an absorbent article of claim 27, wherein the non-target areas of the retention layer are selectively calendered with respect to the target area.

29. An absorbent core for an absorbent article, comprising:
a) a retention layer having major plane surfaces, a thickness, and a target area for positioning in the absorbent article to receive insult from bodily fluids;
b) the retention layer having a horizontal density gradient from a lower density of superabsorbent material in the target area to a higher density of superabsorbent material distal from the target area; and
c) the retention layer being contacted by an upper fluid acquisition and distribution layer and a lower fluid acquisition and distribution layer on its major plane surfaces.

30. The absorbent core of claim 29 wherein the superabsorbent material gradient includes a gradient of amount in the superabsorbent material.

31. The absorbent core of claim 30 wherein the superabsorbent material gradient includes a gradient of type in the superabsorbent material.

32. The absorbent core of claim 29 wherein the superabsorbent material gradient includes a gradient of type in the superabsorbent material.

33. The absorbent core of claim 30 wherein the retention layer further includes a pulp material having a gradient.

34. The absorbent core of claim 33 wherein the pulp material gradient includes a gradient of type in the pulp material.
